(19) United States
(12) Patent Application Publication
(10) Pub. No.: US 2015/0320772 A1
(43) Pub. Date: Nov. 12, 2015

(54) USE OF ACETYLSALICYLIC ACID TO PREVENT AND/OR TREAT DIABETIC WOUNDS

(71) Applicants: LABORATOIRES URGO, Chenove (FR); INSERM (INSTITUT NATIONAL DE LA SANTÉ ET DE LA RECHERCHE MÉDICALE, Paris (FR); INSTITUT DE RECHERCHE POUR LE DÉVELOPPEMENT (IRD), Marseille 2 (FR); UNIVERSITE PAUL SABATIER TOULOUSE III, Toulouse (FR)

(72) Inventors: Agnes Coste, Blagnac (FR); Christophe Dardenne, Fontenilles (FR); Bernard Pipy, Toulouse (FR)

(73) Assignees: LABORATOIRES URGO, CHENOVE (FR); INSERM, Paris (FR); Institut de Recherche pour le Développement (IRD), MARSEILLE (FR); Université Paul Sabatier Toulouse III, TOULOUSE (FR)

(21) Appl. No.: 14/652,912

(22) PCT Filed: Dec. 18, 2013
(86) PCT No.: PCT/FR2013/053155

§ 371 Date: Jun. 17, 2015

(30) Foreign Application Priority Data
Dec. 21, 2012 (FR) 12 62644

(51) Int. Cl. A61K 31/616

(52) U.S. Cl. CPC A61K 31/616

(57) ABSTRACT

The present invention relates to acetylsalicylic acid or a salt thereof, at a concentration comprised between 200 and 4800 μM, for use in the prevention and/or treatment of diabetic wounds.

The subject of the present invention consists of a novel topical use of acetylsalicylic acid at a concentration of between 200 and 4800 μM, for preventing and/or treating diabetic wounds.

Wound healing is a natural physiopathological process, with human and animal tissues being able to repair localized lesions by their own repair and regeneration processes.

Natural wound healing mainly occurs according to three major chronological sequences. Each of these sequences is characterized by specific cellular activities and is controlled by a multitude of regulatory signals (both positive and negative) which collectively manage and support the progression of the repair process. Thus, the following are distinguished:
the inflammatory phase;
the proliferative phase (which comprises the granulation phase and the epithelialization phase); and
the remodeling phase.

The first phase, which is the inflammatory phase, begins as soon as blood vessels are burst, an event which triggers the formation of a clot (blood coagulation) that is mainly composed of fibrin and fibronectin and that will constitute a provisional matrix. This matrix in part fills the lesion and enables the migration, within the damaged area, of the inflammatory cells recruited to ensure detersion of the wound. The platelets present also release factors (for example cytokines and/or growth factors) enabling cells involved in the healing process to be recruited. This phase is characterized by infiltration and activation of numerous inflammatory cells (polymorphonuclear cells, macrophages) at the site of the lesion, which defend the organism against any foreign microorganisms and also clean or deterge the wound.

The second phase corresponds to the development of granulation tissue. First of all, colonization of the injury by migration and proliferation of fibroblasts is observed. Then, the migration of endothelial cells from healthy vessels allows neovascularization, or angiogenesis, of the damaged tissue. In the granulation tissue, fibroblasts are activated and differentiate into myofibroblasts with significant contractile properties provided by actin microfilaments, thus enabling wound contraction. The microfilaments are expressed via a protein, α-smooth muscle actin. These myofibroblasts play an important role in the formation and contraction of the granulation tissue which leads to healing of the lesion. Keratinocytes then migrate from the edges of the wound and then differentiate, leading to reconstruction of the epidermis.

This phase of development of the granulation tissue is initiated following prior reduction in the general state of inflammation of the lesion, gradual disappearance of polymorphonuclear neutrophils and appearance of macrophages, including “repair” macrophages. This transition from the inflammatory phase to the proliferation/repair phase is known as the resolution phase of inflammation.

The third phase of the process is a mainly remodeling stage with the goal of reconstructing a functional tissue, so that the newly formed tissue takes on the initial characteristics and properties of the original tissue. Part of the extracellular matrix is digested by proteases (essentially matrix metalloprotease and elastases), and reorganization of the extracellular matrix is observed. Type III collagen, which is predominant within the granulation tissue, is gradually replaced by type I collagen which is the main matrix component of the dermis. At the end of the maturation phase, the fibroblasts, myofibroblasts and vascular cells experience reduced proliferation and/or activity. Then, the excess cells die through apoptosis, with concomitant remodeling of the extracellular matrix.

Nonetheless, some types of wounds do not heal correctly, with the 3 key stages in the process occurring abnormally despite the best possible physiochemical and biological conditions having been established. Indeed, the speed and quality of wound healing depend on intrinsic and extrinsic factors. This repair process may therefore be prolonged abnormally as a function of:
the etiology of the wound;
the state and location thereof;
the occurrence of an infection caused by the presence of certain infectious agents such as Staphylococcus aureus or Pseudomonas aeruginosa;
the existence of a pre-existing pathological condition (such as diabetes, immunodeficiency, venous insufficiency, etc.);
the external environment; or
genetic factors predisposing or not predisposing to healing disorders.

Among these wounds are chronic wounds such as venous ulcers, bed sores or wounds characteristic of diabetic subjects, and more particularly diabetic foot wounds.

Chronic wounds are defined by an absence of healing after a period of 6 weeks starting from the appearance of the wound, irrespective of the treatment applied. Diabetic wounds are characterized as a specific type, separate from chronic wounds.

Indeed, the primary cause of the absence of healing of these diabetic wounds is associated with an increased bioavailability of glucose. This brings about numerous physiological and metabolic alterations such as thickening of the skin, or significant oxidative stress leading to neuropathy and arteriopathy. Arteriopathy and neuropathy are two major risk factors for chronification and thus delaying of the healing of diabetic wounds, and more particularly diabetic foot wounds. The most well known other types of chronic wounds, such as bed sores or venous, arterial or mixed ulcers, do not follow from the same pathology. For example, venous insufficiency is the cause of the formation, chronification and hence delayed healing of venous ulcers. Bed sores, for their part, are wounds which arise after cycles of ischemia and reperfusion following excessive and prolonged pressure and friction on cutaneous tissues.

It is in this way that all diabetic patients suffering from wounds are exposed to complications which are quite often the cause of the morbidity and mortality of said patient. Several major problems disrupt the correct healing sequence in this type of subject. A first delay in healing occurs from the inflammatory phase: passage from the inflammatory phase to the proliferative phase is disrupted. This is referred to as a problem in the resolution of inflammation. The inflammatory phase is an essential phase in healing, but it must be temporary. Resolution of inflammation is a critical point which conditions the initiation of the other phases of healing. This dynamic event involves the disappearance of the inflammatory cells (polymorphonuclear neutrophils) and the appearance of macrophages. Then, chemotactic and angiogenic anti-inflammatory mediators are produced, which notably enable the migration and differentiation of fibroblasts, which are key cells in the granulation phase.

A disruption to this inflammatory phase, as is the case in the aforementioned subjects, causes an abnormal lengthening of the inflammatory phase and gives rise to chronicity of the wound, thereby delaying all the subsequent stages of healing.

Finally, the phase of wound closure notably during epithelialization is delayed or even, in the majority of cases, does not occur.

It has been known for a long time that aspirin, or acetylsalicylic acid, is considered to be an effective remedy for a wide variety of symptoms or pathological conditions. Its most widespread mode of action is a long-established activity aiming to relieve pain or else reduce fever. It is also used in low doses for its preventative action against the onset of cardiovascular disorders (antithrombotic action).

Indeed, the aggregation-inhibiting properties make it possible to effectively combat the formation of blood clots in the blood vessels. However, it would appear that this activity is more beneficial within the context of a preventative act following a first vascular disorder rather than as primary prevention, where the real effectiveness of this substance seems elusive.

In each of the cases mentioned above, it is a question here of oral administration of aspirin. Applications of aspirin by the topical route also exist. Indeed, aspirin via topical application is also known to have a beneficial action on hypertrophic scars and notably on improving the appearance of these scars (cf. Wound Repair Regen., 2012 Nov. 5., Epub ahead of print, Topical application of a film-forming emulgel dressing that controls the release of stratifin and acetylsalicylic acid and improves/prevents hypertrophic scarring, by Rahmani-Neishaboor E, Jallili R, Hartwell R, Leung V, Carr N, Ghahary A, Department of Surgery, Division of Plastic Surgery, University of British Columbia, Vancouver, BC, Canada).

Moreover, the positive action of acetylsalicylic acid or aspirin or a derivative thereof on the healing of a normal wound and on wound pain is also known, regardless of its aggregation-inhibiting activity, which could seem contradictory to the intended pro-healing action.

Indeed, patent application EP 1 581 252 from the company Kimberly Clark describes a composition comprising aspirin for stimulating cell proliferation, and more particularly fibroblast and keratinocyte proliferation. This composition improves wound healing by stimulating cell proliferation. This document reveals that the effect of aspirin is dose-dependent for concentrations of between 10⁻⁵ and 10⁻⁶ M, with the most effective doses being at the lowest concentrations. The final composition described comprises a concentration of aspirin of between 1 μM and 5 mM. Stimulation of cell proliferation, namely fibroblast and keratinocyte proliferation, and also stimulation of collagen production are important processes in healing, occurring in the proliferative stages and also in the very late phase of remodeling.

For its part, patent application EP 0 784 975 from the Japanese company Teikoku describes an aspirin-based pharmaceutical preparation for topical application, intended to treat skin injuries, mimicked notably in rat models with bed sores. It is noteworthy that these models are healthy animal models. Indeed, none of the rats in the study have any metabolic disorders of the diabetes type. In this application, the aim of the preparation is to increase the speed of formation of granulation tissue and also the speed of restoration of the epidermis in healthy animals with one or more wounds. Thus, this application endeavors to restore the cellular process of healing in its lattermost stages.

None of these documents is concerned with the first stage of healing, namely the inflammatory phase and the resolution thereof, yet the inflammatory phase is an essential and indispensable stage since it initiates the overall healing process. Indeed, diabetic wounds require earlier treatment or intervention in the kinetics of the healing process.

It was for these reasons that the Applicant observed that there was a real need to carry out early, effective and targeted treatment of diabetic wounds.

The Applicant was able to observe, entirely surprisingly, that the use of aspirin enabled a controlled inflammatory phase to be re-established, a phase which could also be described as normal (not prolonged and not aggravated), and also enabled the resolution of inflammation to be accelerated, thereby enabling earlier and quicker wound closure to be achieved in diabetic subjects.

A subject of the present invention is therefore the topical use of acetylsalicylic acid or a salt thereof, at a concentration of between 200 and 4800 μM, for use thereof in preventing and/or treating diabetic wounds.

Thus, a subject of the present invention is acetylsalicylic acid or a salt thereof, at a concentration of between 200 and 4800 μM, for use thereof in preventing and/or treating diabetic wounds.

Another subject of the present invention is a pharmaceutical composition comprising acetylsalicylic acid or a salt thereof, the concentration of which is between 200 and 4800 μM, for use thereof in preventing and/or treating diabetic wounds.

The term “diabetic wound” is intended to mean a wound arising in a diabetic subject with type I or type II diabetes, notably selected from wounds of the lower limbs, and preferably diabetic foot wounds.

The term “salt of acetylsalicylic acid” is intended to mean a salt of acetylsalicylic acid with a pharmaceutically acceptable cation, preferably lysine acetylsalicylate or sodium acetylsalicylate.

Within the context of the present invention, the term “effective dose” is intended to mean a quantity of acetylsalicylic acid or of a salt thereof of between 7.2×10⁻³ and 0.173 mg per wound, preferably of between 7.2×10⁻³ and 0.1044 mg per wound and more preferably between 1.08×10⁻² and 7.2×10⁻² mg per wound. This quantity of acetylsalicylic acid or of a salt thereof is the quantity which is placed in direct contact with the wound. This is a wound which could be defined as having a surface area close to 1 cm². Nonetheless, the effective dose of acetylsalicylic acid or of a salt thereof varies as a function of numerous parameters such as, for example, the size of the wound, and the weight, age, sex, sensitivity and type of individual (human or animal) to be treated. Consequently, the optimal effective dose will have to be determined as a function of the parameters which are judged to be relevant by the specialist in the subject matter.

The term “chronification” is intended to mean any delay in the healing of said wounds.

The term “treating” diabetic wounds is intended to mean healing and closure of said wounds.

The term “preventing” diabetic wounds is intended to mean preventing delayed healing or chronification of said wounds.

Acetylsalicylic acid or a salt thereof is used in the invention at an effective concentration. Indeed, when used at a concentration of less than 200 μM, for example than 100 μM, acetylsalicylic acid or a salt thereof does not have an effect on accelerating the speed of healing of diabetic wounds.

Likewise, at a concentration of greater than or equal to 5000 μM, acetylsalicylic acid no longer has a beneficial action on accelerating the healing of diabetic wounds, and even has a harmful effect on the viability of some cells present in the wound, such as macrophages for example.

According to a preferred embodiment, the acetylsalicylic acid or a salt thereof is used at a concentration of between 200 and 2900 μM.

According to an even more preferred embodiment, the acetylsalicylic acid or a salt thereof is used at a concentration of between 300 and 2000 μM.

Preferably, the defined concentration of acetylsalicylic acid or a salt thereof is the concentration dispensed on contact with the wound.

Preferably, the acetylsalicylic acid or a salt thereof is used for treating the chronification of diabetic wounds. In particular, the acetylsalicylic acid or a salt thereof enables a “controlled” (not prolonged and not aggravated) inflammatory phase to be restored, enables the resolution of inflammation to be accelerated, and consequently enables the kinetics of wound closure to be accelerated.

Preferably, the acetylsalicylic acid or a salt thereof according to the invention is formulated in a physiologically acceptable medium, so as to obtain a pharmaceutical composition. The term “physiologically acceptable medium” is intended to mean a medium compatible with the skin, wounds, mucous membranes and integuments.

Preferably, the acetylsalicylic acid or a salt thereof is combined with a lipid or a hyperoxygenated oil or one of their derivatives. Said lipid or said hyperoxygenated oil may be formulated in the pharmaceutical composition already comprising acetylsalicylic acid or a salt thereof, or else may be administered independently of the acetylsalicylic acid or a salt thereof. Preferably, the lipid is an omega-3 or omega-6 fatty acid, or a combination of the two.

The term “omega-3” is intended to mean any compound selected from the nonlimiting list consisting of α-linolenic acid, eicosapentaenoic acid or else docosahexaenoic acid.

The term “omega-6” is intended to mean any compound selected from the nonlimiting list consisting of linoleic acid, γ-linolenic acid, eicosadienoic acid, dihomo-γ-linolenic acid, arachidonic acid, docosadienoic acid, docosatetraenoic acid or else docosapentaenoic acid.

The term “hyperoxygenated” oil is intended to mean any hyperoxygenated oil of plant origin, the definition of this oil by UV spectrophotometry being from 10 to 20 for factor E270 and from 8 to 60 for factor E232.

Preferably, the hyperoxygenated oil of plant origin has a degree of peroxidation of between 30 and 300 expressed as milliequivalents per kilogram and a content of oxidized glycerides of between 5 and 40.

Preferably still, the hyperoxygenated oil of plant origin has a degree of peroxidation of between 50 and 150 expressed as milliequivalents per kilogram.

According to one preferred embodiment, the hyperoxygenated oil of plant origin is selected from the group consisting of corn oils, sweet almond oils, safflower oils, hazelnut oils, peanut oils, olive oils, rapeseed oils, soybean oils, evening primrose oils, sunflower oils, grapeseed oils, sesame seed oils, and mixtures thereof, and preferably a corn oil.

The acetylsalicylic acid or a salt thereof, and the pharmaceutical composition comprising them, are preferably administered locally, in other words via the topical route. Administration via the local or topical route involves the active ingredient, in this instance acetylsalicylic acid or a salt thereof, acting locally after absorption by the skin, the wound or the mucous membranes. Various pharmaceutical forms exist, such as solutions, emulsions, creams, ointments, lotions, patches, gels, dressings, microcapsules, microfibers, sprays, powders or nanofibers.

Preferably, the acetylsalicylic acid or a salt thereof is included in a (pharmaceutical) composition selected from dressings, microcapsules, microfibers, nanofibers, solutions, lotions, gels, patches, emulsions, creams, ointments, powders and sprays.

When the pharmaceutical composition according to the invention is in the form of an emulsion, it comprises at least one aqueous phase and/or one oily phase, and a surfactant. Indeed, conventional emulsions are unstable, quasi-homogeneous systems of two immiscible liquids, one of which is dispersed in the other in the form of small droplets (micelles). This dispersion is stabilized by virtue of the action of surfactants which modify the structure and the ratio of interfacial forces, and therefore increase the stability of the dispersion by reducing the interfacial tension energy.

The pharmaceutical composition according to the invention may also be in gel form; in this case it comprises one or more gelling compounds.

When the pharmaceutical composition is in the form of a solution, it comprises, other than the acetylsalicylic acid or a salt thereof, an aqueous or oily solution and optionally one or more solvents and/or propenetrating agents for the acetylsalicylic acid or a salt thereof.

The pharmaceutical composition according to the invention may also comprise inert additives or combinations of these additives, such as:
thickening agents or emulsifiers;
pH-regulating agents;
UV-A and UV-B screening agents; and
antioxidants.

The thickening agents or emulsifiers may be selected from the nonlimiting list of compounds comprising notably xanthan gum, or else the surfactant family.

The pH-regulating agents may be selected from the nonlimiting list of compounds comprising notably carbonates.

The UV-A and UV-B screening agents may be selected from the nonlimiting list of compounds comprising chemical and mineral screening agents. Among the chemical screening agents are octocrylene, benzophenones, drometrizole trisiloxane, or else avobenzone. Among the mineral screening agents are zinc oxide or titanium dioxide.

The antioxidants may be selected from the nonlimiting list of compounds notably comprising tocopheryl acetate or ascorbic acid.

Of course, those skilled in the art will take care to choose the optional compound(s) to be added to these pharmaceutical compositions such that the advantageous properties intrinsically linked to the present invention are not, or not substantially, altered by the intended addition.

These additives may be present in the composition at from 0.001 to 20% by weight relative to the total weight of the composition.

The following examples will now be given by way of nonlimiting illustration.

Other features and advantages of the invention will become more clearly apparent upon reading the following description of a preferred embodiment of the invention, given by way of example, to which embodiment the following figure legends refer:

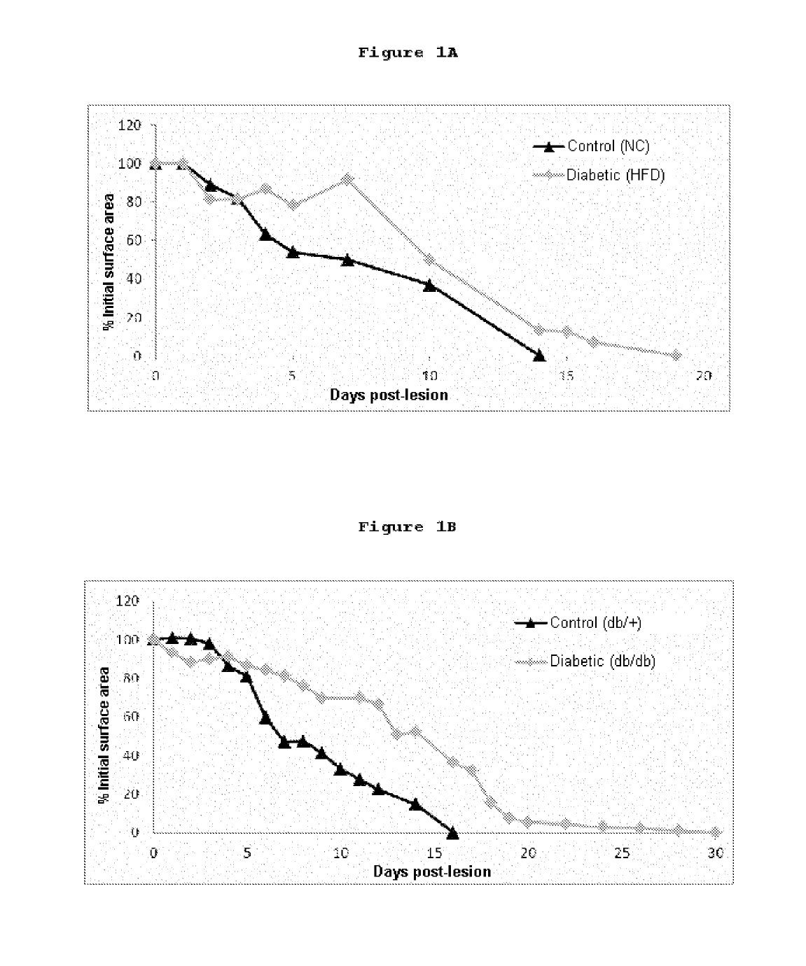

FIG. 1: Kinetics of wound closure in “control” mice and diabetic mice (HFD and db/db)

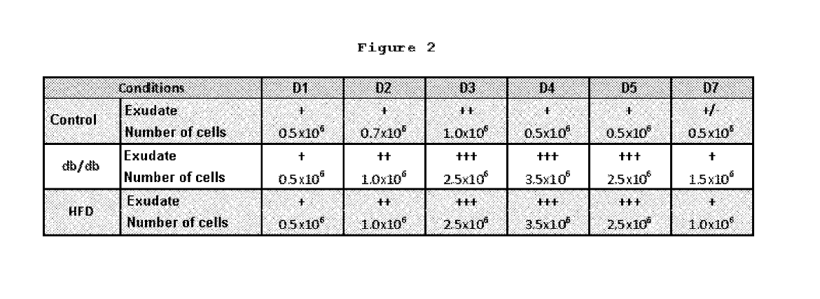

FIG. 2: Evaluation of the exudate and quantification of number of cells in the exudate from “control” mice and diabetic mice (HFD and db/db). The legend for the table is as follows:

Absence of exudate: −

Weeping: +/−

Weak exudation: +

Moderate exudation: ++

Strong exudation: +++

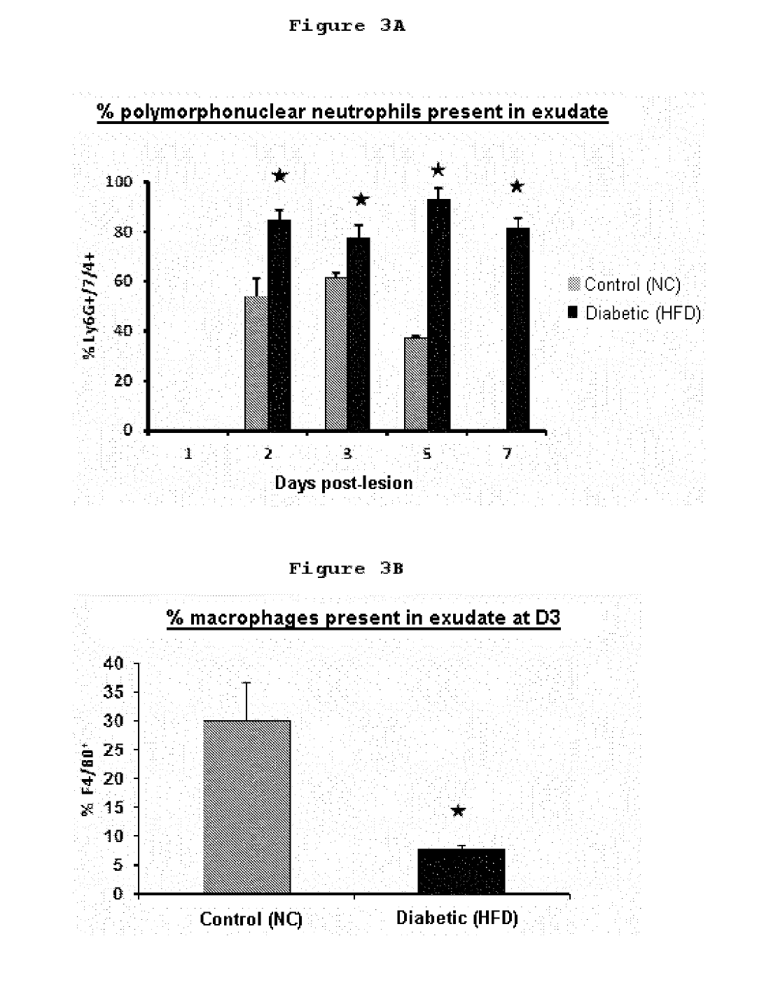

FIG. 3: Quantification of total number of polymorphonuclear neutrophils and macrophages in the exudate from “control” mice and diabetic mice (HFD)

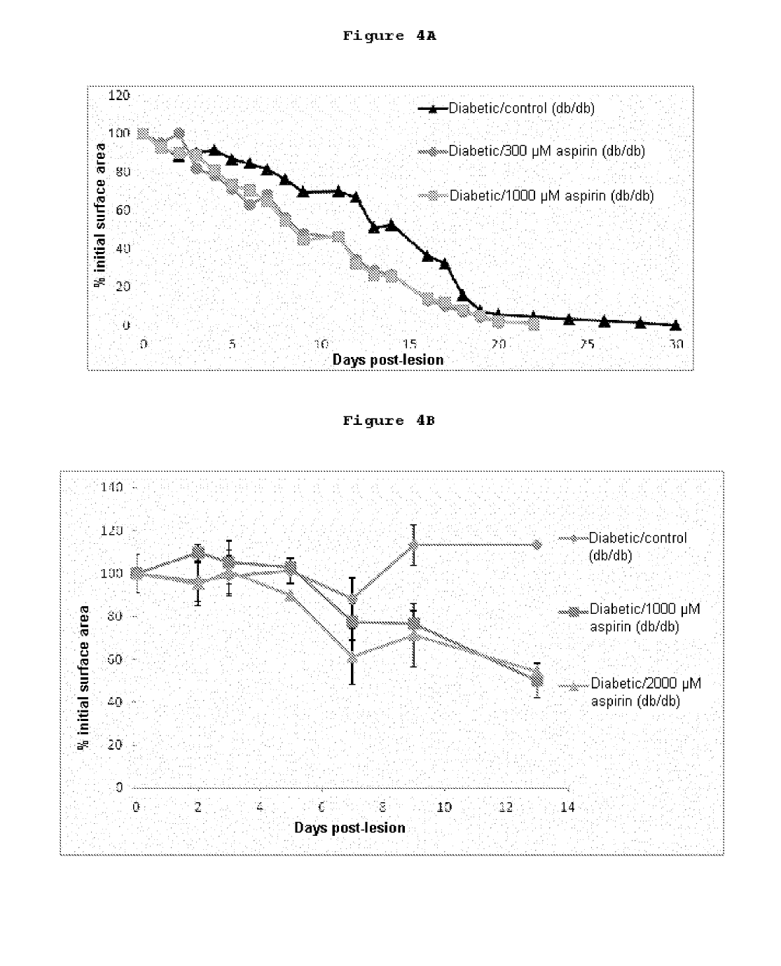

FIG. 4: Kinetics of wound closure in “control” mice and diabetic mice (db/db) treated with acetylsalicylic acid

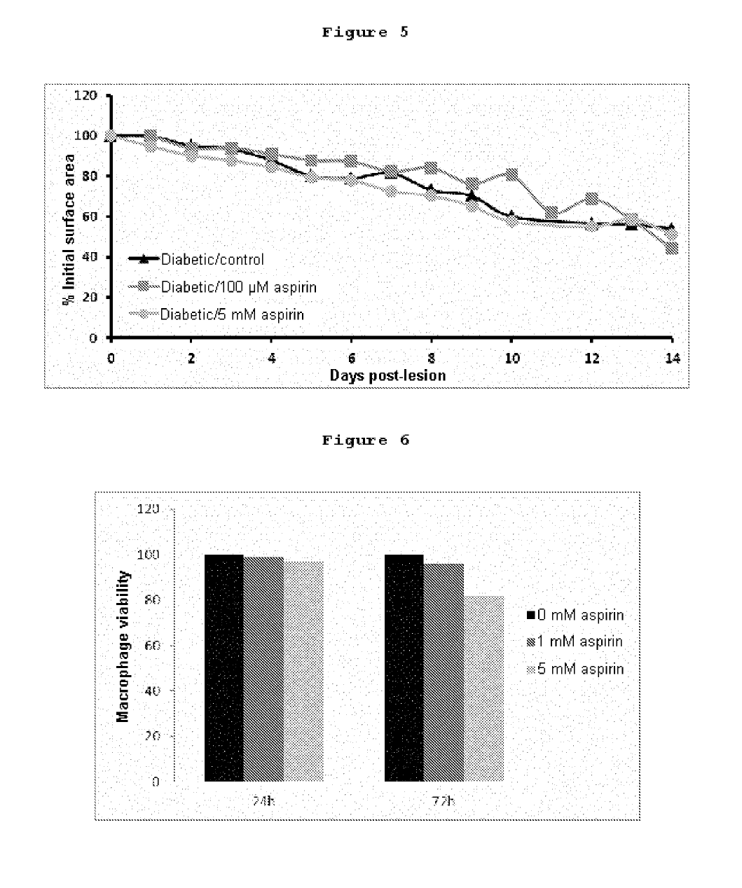

FIG. 5: Kinetics of wound closure in “control” mice and diabetic mice (db/db) treated with acetylsalicylic acid

FIG. 6: Viability of macrophages in vitro in the presence of acetylsalicylic acid

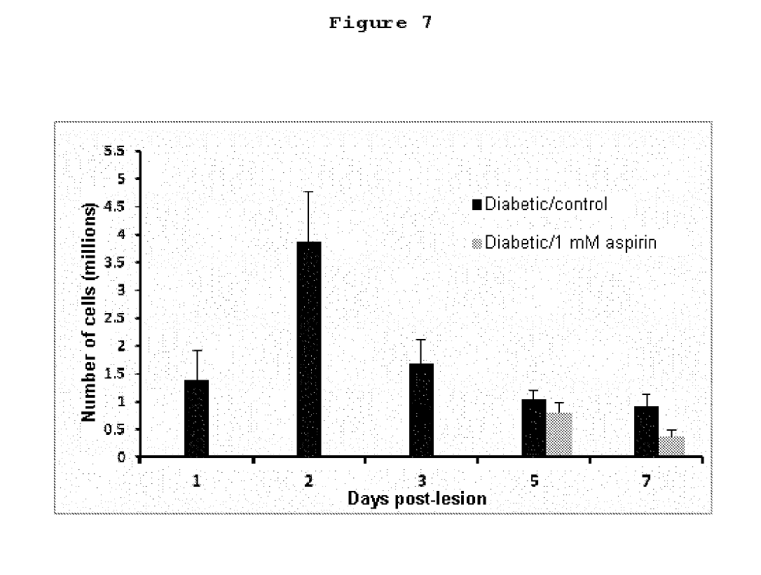

FIG. 7: Kinetics of cell infiltration into the wound of “control” mice and diabetic mice (HFD) treated with acetylsalicylic acid.

### EXAMPLES

### Materials & Methods

Db/Db Mice:

Homozygous db/db mice have a point mutation in the leptin receptor gene, leptin being a hormone which notably controls the sensation of fullness. db/db mice become hyperphagic and life. These animals are characterized by hyperglycemia, insulin resistance and hypertriglyceridemia, which makes these mice the first choice as models for diabetes of genetic origin. The mice of the “control” group for the db/db mice are heterozygous db/+mice.

HFD Mice:

HFD mice are murine models for induced diabetes, produced with C57BL/6 mice of 8 weeks of age. These mice follow a high calorie (high-fat) diet for 16 weeks. They exhibit a high weight gain due to an increase in their adipose tissue, and also develop glucose intolerance and insulin resistance. Unlike db/db mice, the diabetic state in these animals is not of genetic origin. The mice of the “control” group for the HFD mice are mice on a standard diet, also known by the abbreviation “NC”.

All the animals are kept in a daily cycle divided into 12 hours of darkness followed by 12 h of light at 22° C. with constant access to food and water. The establishment of diabetes in the mice is validated by two tests enabling glucose metabolism to be evaluated: the glucose tolerance test (IPGTT) and the insulin sensitivity test (IPIST).

Monitoring Healing Kinetics:

The mouse is anesthetized by means of isoflurane (halogenated anesthetic gas). The dorsal part and the flanks of the mouse are shaved and disinfected and a circular excision of close to 1 cm² is made. The connective tissue at the interface of the muscle tissue is removed without damaging the muscle fascia. The skin is totally excised as far as the panniculus carnosus.

The wound thus created is protected by a device such as described in the patent applications filed by Laboratoires Urgo, FR 11 62295 and FR 11 62344, which enables both protection of the wound and observation and evaluation of the healing kinetics, and enables exudate to be collected.

The wound healing process is observed from 0 to 30 days post-lesion. Between 3 and 5 very high resolution standardized digital photographs are taken for each animal. The percentage wound closure is based on changes in surface area calculated for the same injury on the same mouse and for the indicated time points. Thus, the mean calculated for wound healing (as a percentage) over the 3 mice in a group is obtained independently and the statistics are calculated from 3 groups of 3 independent experiments.

The cells from the exudate are recovered from D1 post-lesion until no more exudate is secreted by the wound. In order to determine the kinetics of polymorphonuclear neutrophil infiltration, the cells from the exudate from the lesion are characterized by flow cytometry by immunolabeling relating to the membrane receptors Ly6G, 7/4 (for the polymorphonuclear cells) and F4/80 (for the macrophages). A total of 10 000 events is analyzed for each sample. All the results are given in the form of percentage expression.

### Results Obtained

The results are expressed as a mean±SEM. SEM denotes the standard deviation from the mean. It represents the deviation from the mean of the sample relative to the mean of the true population. It is calculated by dividing the standard deviation by the square root of the sample size. The statistical analyses are carried out according to the multiple comparisons method, the Bonferroni-Dunnet test relating to at least 3 subjects for each group (n=3).

P<0.05 (*) is considered to be statistically significant.

### A. Monitoring Healing

Kinetics of Wound Closure (FIGS. 1A and 1B):

In the “control” mice, total closure of the lesion is observed in 14 days. On the other hand, in the HFD mice, this total closure is only achieved after a period of 18 days, i.e. an effective 4 day delay (FIG. 1A).

In the db/db mice, the wound is only effectively totally closed between D26 and D28, i.e. with a delay to healing relative to their respective control of 10 to 12 days (FIG. 1B).

There is therefore a significant delay in wound closure in the diabetic mice (HFD and db/db).

Macroscopic Observation of Healing (Exudate+Granulation Tissue) (FIG. 2):

In the “control” mice, following the creation of the cutaneous lesion, laying down of granulation tissue which gradually appears at the wound is observed. The expansion thereof is not homogeneous but originates at various points at the edges of the wound to fill it entirely in 5 days. This tissue thickens as healing progresses.

The diabetic mice exhibit a net delay in the laying down of this granulation tissue from the second day post-lesion. Total filling of the lesion only occurs after 7 days post-lesion in the diabetic mice, i.e. two additional days in comparison to the “control” mice.

In the diabetic mice, the laying down of the granulation tissue is accompanied by a phenomenon of exudation. This liquid, which is rich in cells and mediators, is produced as soon as the lesion is created. In the “control” mice, the peak production is reached from D3 post-lesion.

On the other hand, the diabetic mice (HFD and db/db) exhibit much greater exudation in terms of volume from D2 post-lesion, which especially persists over time (FIG. 2).

There is therefore a significant delay in laying down the granulation tissue in diabetic mice, accompanied by greater and more persistent exudation.

### B. Analysis of Cell Populations Present in the Exudate (FIGS. 2, 3A and 3B)

The “control” mice generally have a low volume of exudate and the cell infiltration in this exudate is limited. Cell infiltration in the exudate is observed on the second day post-lesion up to 5 days post-lesion. Peak cell infiltration is observed on D3 post-lesion (FIG. 2).

The kinetics of cell infiltration in the exudate of the diabetic mice is different than that observed in the “control” mice. Indeed, greater cell infiltration which persists over time up to D7 post-lesion is observed (FIG. 2).

The difference in cell infiltration which is observed as a function of time is explained by the persistence of the polymorphonuclear neutrophils at the site of the lesion in the diabetic mice. Indeed, in the “control” mice, the polymorphonuclear neutrophils disappear by the end of 5 days post-lesion whereas, in diabetic mice, this cell population persists over time, up to D7 post-lesion (FIG. 3A). Thus, it is clearly apparent that the percentage of polymorphonuclear cells infiltrating the cutaneous lesion is much greater for the diabetic group than for the “control” group.

The diabetic mice exhibit a much greater cell infiltration which is caused by the persistence of polymorphonuclear neutrophils at the site of the lesion.

Macrophages arrive at the site of the lesion from D3 post-lesion in the “control” mice. Conversely, this cell infiltration is only poorly visible at D3 post-lesion in the diabetic mice, and does not increase during the continued laying down of the granulation tissue (FIG. 3B).

The diabetic mice exhibit greater cell infiltration which is explained by:
the persistence of polymorphonuclear neutrophils at the site of the lesion;
a delay in recruiting macrophages; and
these polymorphonuclear cells not disappearing.
   Results Obtained in Mice Treated with Acetylsalicylic Acid

### A. Monitoring the Healing Kinetics in Diabetic Mice in the Presence of Acetylsalicylic Acid Used at Various Concentrations

Kinetics of Wound Closure (FIGS. 4, 5 and 6):

As already described above, the mouse is anesthetized by means of isoflurane. The dorsal part and the flanks of the mouse are shaved and disinfected and a circular excision of close to 1 cm² is made. The connective tissue at the interface of the muscle tissue is removed without damaging the muscle fascia. The skin is totally excised as far as the panniculus carnosus.

In the meantime, the reference solution of acetylsalicylic acid is prepared at 5 mM. This solution will be used to prepare the solutions with concentrations of 100 μM, 200 μM, 300 μM, 1 mM, 2 mM, 2.9 mM, 4.8 mM and 5 mM.

Solutions with concentrations of 100 μM, 200 μM, 300 μM, 1 mM, 2 mM, 2.9 mM, 4.8 mM and 5 mM of acetylsalicylic acid are tested.

For reasons of precision, a lookup table for the acetylsalicylic acid concentrations and the doses administered at the wound is established—see below. These doses administered at the wound correspond to a particular embodiment of the invention.

TABLE 1

| Concentration of acetylsalicylic acid | | Dose of acetylsalicylic acid |
|---|---|---|
| tested on the wound | | administered on the wound |
| (for a wound close to 1 cm²) | | (for a wound close to 1 cm²) |
| 100 | μM | 0.0036 mg |
| 200 | μM | 0.0072 mg |
| 300 | μM | 0.0108 mg |
| 1 | mM | 0.036 mg |
| 2 | mM | 0.072 mg |
| 2.9 | mM | 0.1044 mg |
| 4.8 | mM | 0.173 mg |
| 5 | mM | 0.2 mg |

A volume of between 50 and 500 μl is taken off from each solution and placed in contact with the wound via a device such as described through the patent applications filed by Laboratoires Urgo, FR 11 62295 and FR 11 62344.

It is noteworthy that this placing of a predefined concentration of acetylsalicylic acid in contact with the site of healing is carried out starting from the third day post-lesion. Indeed, it is in this period that the delay in healing appears in the diabetic mice, compared to the “control” mice (FIGS. 1A and 1B). Moreover, in the context of treating diabetic patients, the treated wounds will already be formed and will be arrested in the inflammatory phase. No chronic wounds, and more particularly diabetic wounds, are treated as soon as they appear. Moreover, the inflammatory phase is an indispensable phase in healing, since it enables the damaged area to be cleaned and deterged by virtue of the infiltration of inflammatory cells such as polymorphonuclear neutrophils. It is therefore necessary to retain inflammation to initiate effective healing, and thus to not entirely block this phase. This is why treatment with acetylsalicylic acid is only proposed starting from D3 post-lesion.

FIGS. 4A and 4B describe the kinetics of wound healing of diabetic mice following application either of an NaCl solution (corresponding to our “control”) or of a solution of acetylsalicylic acid at 300 μM or at 1000 μM or of a solution at 2000 μM.

The diabetic mice treated with 300 μM, 1000 μM and 2000 μM of acetylsalicylic acid have healing kinetics which are totally different than those of the “control” mice. Whereas in the “control” diabetic mice, wound closure is complete in 26 days, the diabetic mice treated with 300 μM and 1000 μM of acetylsalicylic acid experience wound closure at around twenty days post-lesion. Analogously, FIG. 4B reveals the same results for a dose of 2000 μM.

Moreover, it is possible to observe that the increased speed of closure of the wounds treated with acetylsalicylic acid solutions at 300 μM, 1000 μM and 2000 μM is achieved by means of an acceleration in the early phase of healing (inflammatory phase), namely between D3 and D7 post-lesion, which can be seen in FIG. 4. This acceleration of the healing between D3 and D7 post-lesion correlates to an accelerated speed of resolution of inflammation. Thus, the speed of healing and hence the speed of wound closure, of the mice to which said solutions of acetylsalicylic acid are administered, are greatly accelerated compared to the “control” mice.

In summary, the topical administration of 300 μM, 1000 μM and 2000 μM solutions of acetylsalicylic acid restores a controlled inflammatory phase and induces a very clear acceleration of resolution of inflammation, thereby enabling an increase in the speed of wound closure (at around D20 post-lesion) compared to the “control” diabetic mice.

FIG. 5 describes the kinetics of wound healing of diabetic mice following application either of an NaCl solution (corresponding to our “control”) or of a solution of acetylsalicylic acid at 100 μM or at 5000 μM.

The healing kinetics of the “control” diabetic mice and the diabetic mice treated with 100 μM and 5000 μM of acetylsalicylic acid are similar and do not have any significant differences. No effect whatsoever on the healing kinetics is observed following the introduction of such concentrations of acetylsalicylic acid into contact with the wound, compared with the “control” diabetic mice, especially during the inflammatory phase and during the laying down of the granulation tissue.

In summary, topical administration of solutions of acetylsalicylic acid at 100 μM and 5000 μM to the wounds of diabetic mice shows no effectiveness in restoring a controlled inflammatory phase or in accelerating resolution of inflammation and thus does not enable an increase in the speed of wound closure.

Moreover, cell viability tests are carried out on macrophages for various concentrations of acetylsalicylic acid (FIG. 6).

This method of analysis comprises the use of human peripheral blood-derived monocytes placed in culture in RPMI medium, to which is added M-CSF at 10 ng/ml, for 24 hours in order to differentiate them into macrophages. Acetylsalicylic acid is added to the culture medium in a final concentration of 1000 μM or 5000 μM.

Cell mortality is analyzed, after 72 hours of incubation, by flow cytometry by measuring the cytoplasmic incorporation of propidium iodide.

The aim of such a cell viability test carried out in vitro is to verify the harmfulness of the dose administered to the cells found in the wound, and preferably in the present case to macrophages, which are considered to be a type of effector cell in the healing of the lesion being studied. Indeed, such a study cannot be carried out on polymorphonuclear neutrophils due to their basal level of in vitro mortality being too high.

FIG. 6 shows a very clear loss of cell viability for a dose of 5000 μM of acetylsalicylic acid administered to the cells in vitro at 72 h, with a viability value which is reduced by 20%, and which can therefore be characterized as harmful.

Conversely, the application of a dose of 1000 μM shows no cell toxicity.

Thus, the solution with a concentration of 5 mM administered to the wound is both ineffective on the healing kinetics (FIG. 5) and also proves harmful to the cells present at the site of healing (FIG. 6).

### B. Analysis of Cell Populations Present in the Exudate (FIG. 7)

FIG. 7 describes the kinetics of cell infiltration carried out on “control” diabetic mice or on those to which has been applied a solution of 1000 μM of acetylsalicylic acid.

At D7 post-lesion, it is noted that treatment with acetylsalicylic acid enables a significant reduction in the number of cells infiltrating the site of the lesion in the treated diabetic mice.

It can therefore be concluded, upon reading all the above results, that the treatment based on aspirin at a concentration of between 100 μM and 4800 μM (administered from D3 post-lesion) enables cell infiltration in diabetic mice to be reduced, a controlled inflammatory phase to be restored, and resolution of inflammation to be accelerated so as to enable, finally, increased speed of wound closure.

Acetylsalicylic acid therefore plays a crucial role in the process of resolution of inflammation and consequently on the speed of wound closure for the diabetic individual.

1. A method for preventing and/or treating diabetic wounds, comprising administering to a subject in need thereof an acetylsalicylic acid or a salt thereof, in a concentration of between about 200 μM and about 4800 μM.

2. The method of claim 1 wherein the acetylsalicylic acid or a salt thereof is in a concentration of between about 200 μM and about 2900 μM.

3. The method of claim 1 wherein the acetylsalicylic acid or a salt thereof is in a concentration of between about 300 μM and about 2000 μM.

4. The method of claim 1 wherein the acetylsalicylic acid or a salt thereof is in a form suitable for topical administration.

5. The method of claim 1 wherein the acetylsalicylic acid or a salt thereof of is selected from the group consisting of lysine acetylsalicylate and sodium acetylsalicylate.

6. The method of claim 1 wherein the acetylsalicylic acid or a salt thereof of is included in a composition selected from the group consisting of dressings, microcapsules, microfibers, nanofibers, solutions and sprays.

7. The method of claim 1 wherein said method is used to treat the chronification of diabetic wounds.

8. The method of claim 1 wherein said method is used for accelerating resolution of inflammation.

9. The method of claim 1 wherein the acetylsalicylic acid or a salt thereof, is combined with a lipid, a hyperoxygenated oil or one of their derivatives.

10. The method of claim 9 wherein the lipid or a derivative thereof is selected from the group consisting of an omega-3 or omega-6 fatty acid, or a combination of the two.

11. A method of preventing and/or treating diabetic wounds comprising administering to a person in need thereof a pharmaceutical composition comprising acetylsalicylic acid or a salt thereof, wherein the acetylsalicylic acid or a salt thereof is in a concentration of between about 200 μM and about 4800 μM, diabetic wounds.
